(19) United States
(12) Patent Application Publication
(10) Pub. No.: US 2005/0059949 A1
(43) Pub. Date: Mar. 17, 2005

(54) Inexpensive absorbent garments

(76) Inventor: Kathy Murguly, Hillsborough, NJ (US)

Correspondence Address: ROPES & GRAY LLP, ONE INTERNATIONAL PLACE, BOSTON, MA, 02110-2624, US

(21) Appl. No.: 10/942,470

(22) Filed: Sep. 16, 2004

Related U.S. Application Data
(60) Provisional application No. 60/503,532, filed on Sep. 16, 2003.

(51) Int. Cl. A61F 13/15

(52) U.S. Cl. 604/387

(57) ABSTRACT

The present invention discloses an absorbent article with a positioning adhesive for attachment, and a method of manufacture. The absorbent article is of a conventional design except that conventional tape systems or hook and loop systems are replaced with positioning adhesive and release paper.

### CROSS-REFERENCE TO RELATED APPLICATIONS

This patent application claims the benefit of, and incorporates by reference in its entirety, prior provisional patent application U.S. Provisional Patent Application No. 60/503,532, filed Sep. 16, 2003.

### BACKGROUND OF THE INVENTION

The marketplace today in the industrialized world provides various types and various priced disposable diapers. In certain parts of the world, non-industrial or third world, most of these types of diapers are unaffordable for the average consumer. Even in the industrial world, there is a segment of the population that would welcome a very low cost alternative to what is presently available as a diaper.

Accordingly, it will be readily appreciated that a need exists for an affordable, low cost, disposable diaper.

### SUMMARY OF THE INVENTION

The present invention discloses an absorbent article with a positioning adhesive for attachment, and a method for manufacturing the absorbent article. The absorbent article is of a conventional design except that conventional tape systems or hook and loop systems can be replaced with a positioning adhesive and release paper.

### BRIEF DESCRIPTION OF THE DRAWINGS

FIG. 1 is a top view of one embodiment of the present invention showing an absorbent garment with a positioning adhesive applied to the right and left ear of the garment.

FIG. 2 is a top view of another embodiment of the present invention showing an absorbent garment with positioning adhesives applied to both the right and left ears (upper and owner) of the garment.

FIG. 3A is a cross-sectional view showing how one embodiment of the present invention can be constructed, wherein a positioning adhesive is applied to the top surface (body side facing) of the absorbent garment, and release paper is applied on top of the adhesive.

FIG. 3B is a cross-sectional view showing how a positioning adhesive is applied to the top surface (body side facing) of the absorbent garment, and the back surface is folded over the adhesive.

### DETAILED DESCRIPTION OF THE INVENTION

The present invention provides a low cost disposable absorbent garment and a method for making such a garment.

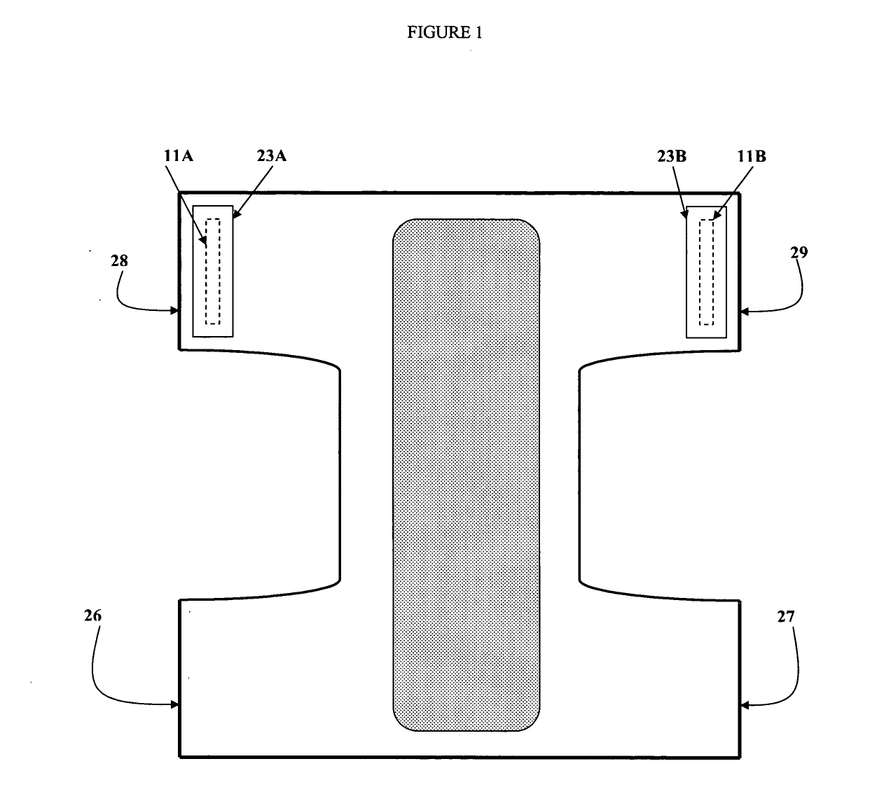

FIG. 1 illustrates the low cost absorbent garment of the present invention. FIG. 1 shows adhesives 11A and 11B applied along a left edge 28 and right edge 29 of the garment. To cover adhesive 11A and 11B prior to its application, release papers 23A and 23B are applied. The release papers 23A and 23B can be removed and discarded when attaching the product to the user. Note that left edge 28 and right edge 29 of the product may be folded over and bonded, in order to create a finger tab area. This would allow easier detachment of each edge of the product in the event that the product needs to be refastened to the user.

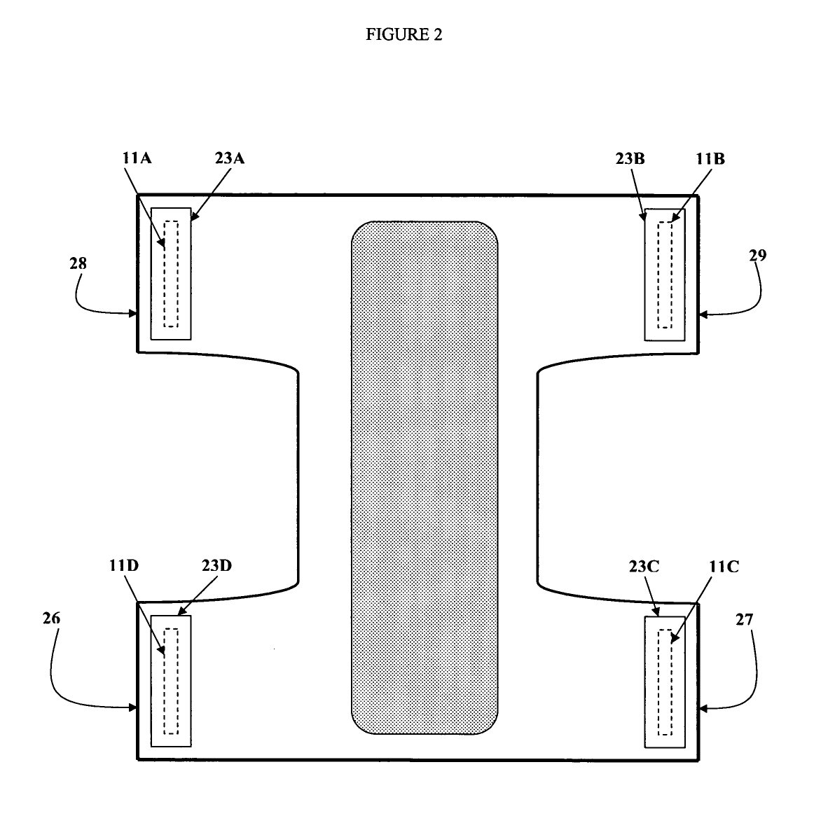

In another embodiment shown in FIG. 2, the low cost absorbent garment of the present invention has adhesives 11A, 11B, 11C and 11D (hereinafter “11”) applied along left edges 28 and 26 and right edges 29 and 27 of the garment. To cover adhesives 11 prior to its application, release papers 23A, 23B, 23C and 23D (hereinafter “23”) are applied. The release papers 23 can be removed and discarded when attaching the product to the user. Note that left edges 28 and 26 and right edges 29 and 27 of the product may be folded over and bonded, in order to create a finger tab area. Having the adhesive attachment on the front and back of the garment gives the care giver or wearer the option of being able to attach the front to the back or vice versus.

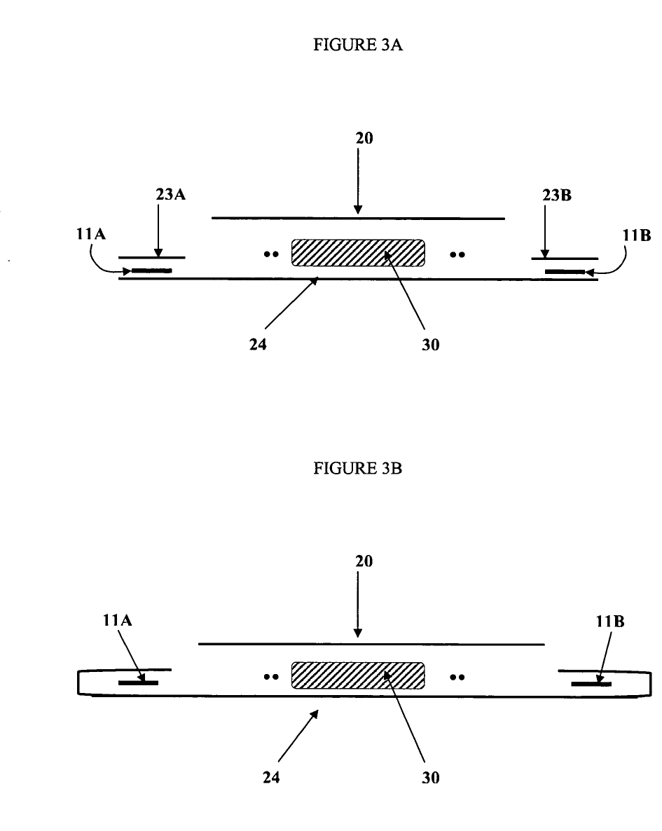

FIG. 3A and FIG. 3B illustrate two alternative ways to construct the low cost absorbent garment of the present invention. Both Figures illustrate that positioning adhesives 11A and 11B are applied along the left and right edges of the absorbent garment. FIG. 3A shows release papers 23A and 23B applied on top of adhesives 11A and 11B. FIG. 3B shows bottom surface 24A and 24B folded over adhesives 11A and 11B. The folded-over section of bottom surface 24 would have a coated surface, such as silicone, in contact with the positioning adhesive 11A and 11B within a zoned area, in order to allow for easy detachment.

The top surface sheet, used to make the low cost absorbent garment of the present invention can be made of various low cost materials. One such material may be a non-woven fabric, such as SBPP (Spundbond Polypropylene) or SMS (Spunbond-Meltblown-Spunbond). Such non-woven materials are commercially available from SAAF Advanced Fabrics of Al-Ahsa, Saudi Arabia. Yet other acceptable materials would be other non-woven fabrics such as ABPET (Adhesive Bonded Polyester), TABICO (Through Air Bonded Bi-Component Fiber), TBPP (Thermal Bonded polypropylene), spunlace fibers, hydro-entangled fibers, needle-punched fibers, airlaid composites and woven fabrics. Non-woven and hydro-entangled materials are commercially available from PGI Nonwovens of Mooresville, N.C.

The bottom surface sheet can comprise a fluid-impervious film such as polyethylene, polypropylene, polyester, co-extruded polymers, synthetic elastics, metallicenes, other polyolefins, etc. In addition, the low cost absorbent garment of the present invention can also comprise a breathable poly film and non-woven fabric, which are laminated together. The breathable poly film may comprise a micro-porous film, 2-dimensional apertured film, 3-dimensional apertured film, hydro-entangled film, or any combination thereof. Such 3-dimensional apertured poly film are commercially available from Tredegar Film Products of Richmond, Va.

A possible acceptable sheet can also be a combination of layers of the materials listed above. Such combinations can be bonded in various ways, such as adhesive, cohesive, ultrasonic bonding, chemical bonding, heat sealing, poly extrusion, etc.

The positioning adhesive used in the side panel of the present invention can be any type known by those skilled in the art. Positioning adhesives are typically referred to as hot melt adhesives that are pressure sensitive. One such positioning adhesive is H2916H available from Bostik Findley of Wauwatosa, Wis. In a preferred embodiment of the invention, the positioning adhesive can be covered by release paper. Release papers are typically coated with silicone on one side and contain a print on the other. One such release paper is available from Tekkote of Leonia, N.J.

In another embodiment of the invention, a release material can be folded over the positioning adhesive. The release material can comprise a silicone, or other releasable material (ie. Teflon), that is coated right on top of the sheet in a zoned area. The zoned area can be situated directly over the positioning adhesive, with some over-lap, after folding-over the edges of the product.

Without further elaboration, the foregoing is intended to be illustrative and not limiting of the invention so others may, by applying current or future knowledge, adopt the same for use under various conditions of service and use.

1. An absorbent article, comprising:
a) a front portion having a right edge and a left edge;
b) an absorbent core; and
c) a back portion having a right edge, left edge and an adhesive attachment means positioned along both the right and left edge,
wherein the front portion of the absorbent article is positioned in front of a wearer, the absorbent core is positioned between the legs of a wearer, and the back portion of the article is positioned in back of the wearer, and wherein the adhesive attachment means is adapted to affix the right edge of the back portion to the right edge of the front portion and the left edge of the back portion to the left edge of the front portion.

2. The absorbent article of claim 1, wherein the adhesive attachment means is positioned on the right edge and left edge of the front portion, allowing for attachment to the right edge and left edge of the back portion.

3. An absorbent article, comprising:
a) a front portion having a right edge and left edge;
b) an absorbent core; and
c) a back portion having a right edge, left edge and an adhesive attachment means positioned along both the right and left edge,
wherein the front portion is positioned in front of a wearer, the absorbent core is positioned between the legs of the wearer and the back portion is positioned in back of the wearer, and wherein the adhesive attachment means is adapted to affix the right edge and back edge of the back portion and front portion, allowing the attachment of the right edge and left edge of the front portion to the right and left edge of the back portion.
